(19) United States
(12) Patent Application Publication
(10) Pub. No.: US 2007/0098800 A1
(43) Pub. Date: May 3, 2007

(54) Therapeutic compositions

(76) Inventors: Karen J. Giroux, Highland Park, NJ (US); Robert F. Butz, Phoenixville, PA (US)

Correspondence Address: CONNOLLY BOVE LODGE & HUTZ, LLP, P O BOX 2207, WILMINGTON, DE, 19899, US

(21) Appl. No.: 10/532,703

(22) PCT Filed: Oct. 28, 2003
(86) PCT No.: PCT/US03/34183

§ 371 Date: Jan. 19, 2007

Related U.S. Application Data
(60) Provisional application No. 60/421,888, filed on Oct. 28, 2002.

(51) Int. Cl. A61K 9/14; A61K 31/785

(52) U.S. Cl. 424/489; 424/78.27; 424/78.3

(57) ABSTRACT

The present invention provides new formulations of injectable particles (e.g. microspheres) useful for intra-articular (i.a.) injection. The formulations are made of biocompatible polymers that biodegrade to generate NSAIDs, ad are useful for treating inflamed joints, thus providing safe, long-lasting relief of joint pain and swelling. In one embodiment, the present invention provides an injectable particle, comprising a biodegradable polymer comprising an agent selected from the group consisting of an NSAID, a COX-2 inhibitor, an anesthetic and a narcotic analgesic.

### BACKGROUND OF THE INVENTION

Rheumatoid arthritis (RA) is a debilitating disease affecting about 2.1 million Americans between the ages of 20 and 50. By far the most troubling symptoms are severe pain and swelling of the joints of the wrists, hands, ankles and feet, which occur when the body's immune system mistakenly attacks the synovial cells lining the joints, causing intense inflammation (Goronzy, J. J., and Weyand, C. M. Rheumatoid Arthritis: Epidemiology, Pathology, and Pathogenesis, Primer on the Rheumatic Diseases, 12^{th} Ed., Klippel,. J. H., Crofford, L. J., Stone, J. H., Weyand, C. M., Eds. Atlanta: Arthritis Foundation, 2001).

The therapeutic mainstay of RA is oral nonsteroidal anti-inflammatory drugs (NSAIDs) such as aspirin, ibuprofen, fenoprofen, indomethacin, naproxen, diflunisal and others. These drugs produce their anti-inflammatory effects in large part, if not entirely, by inhibiting the cyclo-oxygenase enzymes (COX-1 and COX-2) involved in the production of prostaglandins (Roberts, L. J., Morrow, J. D. Analgesic-Antipyretic and Anti-Inflammatory Agents and Drugs Employed in the Treatment of Gout, Goodman & Gilman's The Pharmacological Basis of Therapeutics, 10^{th} Edition, Hardman, J. G., Limbid, L. E., Gilman, A. G., Eds. New York: McGraw-Hill, 2001). Despite the popularity of newer COX-2 specific NSAIDs like rofecoxib and celecoxcib, many rheumatologists still consider aspirin to be the NSAID of choice for most patients (Simon, L. S. Nonsteroidal Anti-Inflammatory Drugs, Primer on the Rheumatic Diseases. 12^{th} Ed., Klippel,. J. H., Crofford, L. J., Stone, J. H., Weyand, C. M., Eds. Atlanta: Arthritis Foundation, 2001). As disease severity progresses, oral corticosteroids are added to NSAID therapy, but their chronic use can cause bone disorders, cataracts, weight gain, diabetes, and hypertension. In the most debilitating cases, disease-modifying anti-rheumatic drugs (DMARDs) are used, despite their serious side effects. More recently, injectable proteins that block the action of tumor necrosis factor (etanercept and infliximab) or interleukein-1 (anakinra) have been introduced (Lewis, C. Arthritis: Timely Treatments for an Ageless Disease, FDA Consumer Magazine. Bethesda: U.S. Food and Drug Administration, 2000).

Notwithstanding the effectiveness of these treatments, RA remains a chronic disease, the severity of which fluctuates over time. When pain and swelling flare, a standard treatment is to inject corticosteroids directly into the affected joint, sometimes in combination with a local anesthetic. Such intra-articular (i.a.) injections provide rapid and long-lasting relief of pain and swelling, but only a few steroid injections can be administered safely at any one time, and repeated injections into the same joint can destroy cartilage (Stefanich, R. J. Intra-articular corticosteroids in treatment of osteoarthritis. Orthoped. Rev. 1986 32:65-71); Kongtawelert, P., Brooks, P., Ghosh, P. Pentosan polysulfate (Cartrophen) prevents the hydrocortisone-induced loss of hyaluronic acid and proteoglycans from cartilage of rabbit joints as well as normalizes the keratan sulfate levels in their serum. J. Rheumatol. 1989 16:1454-1459).

These drawbacks have spurred the development of “steroid-sparing” treatments for flared joints. One such attempt involves the steroid, betamethasone, formulated for i.a. injection with poly(lactic acid-co-glycolic acid) (PLGA) microspheres, with the goal of slowing the delivery of betamethasone to reduce tissue damage (Horisawa, E., Hirota, T., Kawashima, Y. et al. Prolonged anti-inflammatory action of DL-lactide/glycolide copolymer nanospheres containing betamethasone sodium phosphate for an intraarticular delivery system in antigen-induced arthritic rabbit. Pharm. Res. 2002 19:403-410). Another attempt involves narcotic analgesics. Long considered to produce analgesia by the activation of opioid receptors located exclusively within the central nervous system, newer evidence demonstrates that narcotic analgesics such as morphine also produce potent local analgesic effects when injected into chronically-inflamed tissues (Stein, C., Yassouridis, A. Peripheral morphine analgesia. Pain 1997 71: 119-121); Dionne, R. A., Lepinski, A. M., Gordon, S. M., et al. Analgesic effects of peripherally administered opioids in clinical models of acute and chronic inflammation. Clin. Pharmacol. Ther. 2001 70:66-73); Likar, R., Koppert, W., Blatnig, H. et al. Efficacy of peripheral morphine analgesia in inflamed, non-inflamed and perineural tissue of dental surgery patients. J. Pain Symptom Manage, 2001 21:330-337). A clinical study demonstrates that single i.a. injections of small (e.g., 3-mg) doses of morphine provide pain relief similar to 4 mg of dexamethasone in RA patients (Stein, A., Yassouridis, A., Szopko, C. et al. Intra-articular morphine versus dexamethasone in chronic arthritis. Pain 1999 83:525-532).

In spite of the above reports, there remains a need for suitable steroid-sparing i.a. injectable treatments for RA. There is also a need for suitable steroid-sparing treatments for this condition that provide long-lasting relief of pain and swelling in inflamed joints, preferably from a single or infrequent injections, that cause less destruction of joint cartilage, and fewer other adverse effects, compared to current therapies.

There is also a need for suitable localized, non-steroidal treatments for other painful conditions that are currently treated using steroid injections including spinal stenosis, bursitis, tendonitis, epicondylitis, fibromyalgia, some forms of chronic foot and ankle pain, calcaneal spur syndrome, some forms of neuralgia, metatarsalgia, metatarsophalangeal articulation, osteoarthritis and others. In addition, steroids are often used to prevent or reduce swelling of central nervous system tissues after brain injuries or in response to viral and other infections. Inflammatory responses of the nervous system and surrounding tissues upon injury have also been shown to inhibit or hinder nerve growth needed to regain normal, functional nervous system connections. Non-steroidal compositions that can be injected to treat these conditions are needed.

### SUMMARY OF THE INVENTION

One or more of the above needs are met by the present invention. The present invention provides new formulations of injectable particles (e.g. microspheres) useful for intra-articular (i.a.) injection. The formulations are made of biocompatible polymers that biodegrade to generate NSAIDs, and are useful for treating inflamed joints, thus providing safe, long-lasting relief of joint pain and swelling. In one embodiment, the present invention provides an injectable particle, comprising a biodegradable polymer comprising an agent selected from the group consisting of an NSAID, a COX-2 inhibitor, an anesthetic and a narcotic analgesic.

The present invention also provides formulations of such particles made of the above polymers into which have been added pharmacologically useful amounts of local anesthetic and/or narcotic analgesic drugs, thus providing additional therapeutic benefit.

Accordingly, the present invention also provides an injectable particle comprising: 1) a biodegradable polymer comprising an NSAID in the polymer backbone; and in combination, 2) one or more NSAIDs, COX-2 inhibitors, local anesthetics or narcotic analgesics.

The invention also provides an injectable particle of the invention which is a microsphere comprising: 1) polymer having a backbone, wherein the backbone comprises one or more groups that will yield an NSAID upon hydrolysis of the polymer; and optionally 2) a local anesthetic or a narcotic analgesic.

The invention also provides a pharmaceutical composition of the invention that is a pharmaceutical composition comprising a plurality of microspheres of the invention and a pharmaceutically acceptable carrier.

The invention also provides a method for treating RA in a mammal comprising administering to the mammal, an effective amount of a microsphere of the invention.

The invention also provides a method for treating RA in a mammal comprising administering to the mammal, an effective amount of a pharmaceutical composition of the invention.

The invention also provides a microsphere of the invention for use in medical therapy.

The invention also provides a composition of the invention for use in medical therapy.

The invention also provides the use of a microsphere of the invention for the manufacture of a medicament useful for the treatment of a RA in a mammal.

The invention also provides synthetic processes disclosed herein that are useful for preparing an injectable particle of the invention.

In one embodiment, the present invention incorporates the discovery that additional therapeutic benefit is provided by the addition to the polymer of a local anesthetic drug and/or a narcotic analgesic drug. The present invention also incorporates the discovery that i.a. injection of a suitable formulation of microspheres made of a biocompatible, biodegradable polymer, alone or containing a local anesthetic drug and/or a narcotic analgesic drug, causes less destruction of joint cartilage, and fewer other adverse effects, compared to i.a injection of a corticosteroid. The present invention also incorporates the discovery that, unlike many other injectable, inhalable, or oral formulations of narcotic analgesic drugs, the formulation of an injectable particle made of a biocompatible, biodegradable polymer containing a narcotic analgesic drug will have minimal potential for abuse.

### DETAILED DESCRIPTION OF THE INVENTION

The following definitions are used, unless otherwise described: halo is fluoro, chloro, bromo, or iodo. Alkyl, alkoxy, etc. denote both straight and branched groups; but reference to an individual radical such as “propyl” embraces only the straight chain radical, a branched chain isomer such as “isopropyl” being specifically referred to. Aryl denotes a phenyl radical or an ortho-fused bicyclic carbocyclic radical having about nine to ten ring atoms in which at least one ring is aromatic. Heteroaryl encompasses a radical attached via a ring carbon of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₆)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

The term ester linkage means —OC(═O)— or —C(═O)O—; the term thioester linkage means —SC(═O)— or —C(═O)S—; and the term amide linkage means —N(R)C(═O)— or —C(═O)N(R)—, wherein each R is a suitable organic radical, such as, for example, hydrogen, (C₁-C₆)alkyl, (C₃-C₆)cycloalkyl, (C₃-C₆)cycloalkyl(C₁-C₆)alkyl, aryl, heteroaryl, aryl(C₁-C₆)alkyl, or heteroaryl(C₁-C₆)alkyl.

The term “amino acid,” comprises the residues of the natural amino acids (e.g. Ala, Arg, Asn, Asp, Cys, Glu, Gln, Gly, His, Ile, Leu, Lys, Met, Phe, Pro, Ser, Thr, Trp, Tyr, and Val) in D or L form, as well as unnatural amino acids (e.g. phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine). The term also comprises natural and unnatural amino acids bearing a conventional amino protecting group (e.g. acetyl or benzyloxycarbonyl), as well as natural and unnatural amino acids protected at the carboxy terminus (e.g. as a (C₁-C₆)alkyl, phenyl or benzyl ester or amide; or as an α-methylbenzyl amide). Other suitable amino and carboxy protecting groups are known to those skilled in the art (See for example, Greene, T. W.; Wutz, P. G. M. “Protecting Groups In Organic Synthesis” second edition, 1991, New York, John Wiley & sons, Inc., and references cited therein).

The term “peptide” describes a sequence of 2 to 35 amino acids (e.g. as defined hereinabove) or peptidyl residues. The sequence may be linear or cyclic. For example, a cyclic peptide can be prepared or may result from the formation of disulfide bridges between two cysteine residues in a sequence. Preferably a peptide comprises 3 to 20, or 5 to 15 amino acids. Peptide derivatives can be prepared as disclosed in U.S. Pat. Nos. 4,612,302; 4,853,371; and 4,684,620, or as described in the Examples herein below. Peptide sequences specifically recited herein are written with the amino terminus on the left and the carboxy terminus on the right.

A “narcotic analgesic” is any analgesic that produces a narcotic effect. The term “narcotic analgesic” also includes any habit-forming drug, such as, e.g., opiates such as, for example, morphine and heroin; opioids, such as, e.g., synthetic drugs such as meperidine (Demerol).

Polymers

Biocompatible, biodegradable polymers suitable for use in the present invention include all biodegradable polymers that are suitable for administration to a mammal and that are capable of acting as a carrier for a pharmaceutically active substance such as an NSAID, a narcotic analgesic, or a local anesthetic. For Example, see Erdmann, L., Uhrich, K. E., Biomaterials, 2000, 21:1941-1946. Suitable polymers are also described in, e.g., U.S. Pat. Nos. 6,328,988; 6,365,146; 6,468,519; 6,486,214; 6,497,895; 6,602,915; 6,613,807; U.S. Published Patent Applns. 2002/0071822 A1; 2002/0106345 A1; 2003/0035787 A1; 2003/0059469 A1; 2003/0104614 A1; 2003/0170202 A1; U.S. patent application Ser. Nos. 09/508,217; 10/368,288; 10/622,072; 10/646,336; 10/647,701; and International Patent Applns. WO 99/12990; WO 01/28492; WO 01/41753; WO 01/58502; WO 02/09767; WO 02/09768; WO 02/09769; WO 03/005959; WO 03/046034; WO 03/065928; and WO 03/072020.

Biocompatible, biodegradable, anti-inflammatory polymers suitable for use in the present invention include, but are not limited to, polymers described by Erdmann, L., Uhrich, K. E., Biomaterials, 2000, 21:1941-1946.

Biocompatible, biodegradable, anti-inflammatory polymers suitable for use in the present invention also include, but are not limited to, polymers described in International Patent Application Publication Number WO 02/09768A2. For example, a suitable polymer is a polymer that comprises one or more units of formula (I):
—R₁-A-L-A- (I)

in the polymer backbone, wherein: R₁ is group that will provide an NSAID upon hydrolysis of the polymer; each A is independently an amide linkage, a thioester linkage, or an ester linkage; and L is a linking group. Another suitable polymer is a polymer that comprises one or more units of formula (II) in the backbone:
—R₂-A-L-A-R₃-A-L-A- (II)

wherein: R₂ and R₃ are each independently a group that will yield an NSAID upon hydrolysis of the polymer; each A is independently an amide, thioester, or ester linkage; and each L is independently a linking group.

Biocompatible, biodegradable, anti-inflammatory polymers suitable for use in the present invention also include, but are not limited to, polymers described in International Patent Application Publication Number WO 02/09767A2. For example, a suitable polymer is a polymer that comprises a backbone, wherein the backbone comprises one or more anhydride linkages, and wherein the backbone comprises one or more groups that will yield an NSAID upon hydrolysis of the polymer. Another suitable polymer is a polymer that comprises one or more units of formula (III) in the backbone:
—C(═O)R⁴—X-L-X—R⁴—C(═O)—O— (III)

wherein: each R⁴ is group that will provide an NSAID upon hydrolysis of the polymer; each X is independently an amide linkage, a thioester linkage, or an ester linkage; and L is a linking group.

Biocompatible, biodegradable, anti-inflammatory polymers suitable for use in the present invention also include, but are not limited to, polymers described in International Patent Application Publication Number WO 99/12990. For example, a suitable polymer is a polymer described therein that will yield an NSAID upon hydrolysis of the polymer.

Linking Group “L”

The nature of the linking group “L” in a polymer is not critical provided the polymer possesses acceptable mechanical properties and release kinetics for the selected therapeutic application. The linking group L is typically a divalent organic radical having a molecular weight of from about 25 daltons to about 400 daltons. More preferably, L has a molecular weight of from about 40 daltons to about 200 daltons.

The linking group L typically has a length of from about 5 angstroms to about 100 angstroms using standard bond lengths and angles. More preferably, the linking group L has a length of from about 10 angstroms to about 50 angstroms.

The linking group may be biologically inactive, or may itself possess biological activity. The linking group can also comprise other functional groups (including hydroxy groups, mercapto groups, amine groups, carboxylic acids, as well as others) that can be used to modify the properties of the polymer (e.g. for branching, for cross linking, for appending other molecules (e.g. another biologically active compound) to the polymer, for changing the solubility of the polymer, or for effecting the biodistribution of the polymer).

Specific And Preferred Values

Specific and preferred values listed herein for radicals, substituents, groups, and ranges, are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₁-C₆)alkanoyl can be acetyl, propanoyl or butanoyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutylthio, pentylthio, or hexylthio; (C₂-C₆)alkanoyloxy can be acetoxy, propanoyloxy, butanoyloxy, isobutanoyloxy, pentanoyloxy, or hexanoyloxy; aryl can be phenyl, indenyl, or naphthyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

A specific value for L is a divalent, branched or unbranched, saturated or unsaturated, hydrocarbon chain, having from 1 to 25 carbon atoms, wherein one or more (e.g. 1, 2, 3, or 4) of the carbon atoms is optionally replaced by (—O—) or (—NR—), and wherein the chain is optionally substituted on carbon with one or more (e.g. 1, 2, 3, or 4) substituents selected from the group consisting of (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo (═O), carboxy, aryl, aryloxy, heteroaryl, and heteroaryloxy.

Another specific value for L is a divalent, branched or unbranched, saturated or unsaturated, hydrocarbon chain, having from 1 to 25 carbon atoms, wherein the chain is optionally substituted on carbon with one or more (e.g. 1, 2, 3, or

4) substituents selected from the group consisting of (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo, carboxy, aryl, aryloxy, heteroaryl, and heteroaryloxy.

Another specific value for L is an amino acid.

Another specific value for L is a peptide

Another specific value for L is a divalent, branched or unbranched, saturated or unsaturated, hydrocarbon chain, having from 1 to 25 carbon atoms, wherein one or more (e.g. 1, 2, 3, or 4) of the carbon atoms is optionally replaced by (—O—) or (—NR—).

A more specific value for L is a divalent, branched or unbranched, saturated or unsaturated, hydrocarbon chain, having from 3 to 15 carbon atoms, wherein one or more (e.g. 1, 2, 3, or 4) of the carbon atoms is optionally replaced by (—O—) or (—NR—), and wherein the chain is optionally substituted on carbon with one or more (e.g. 1, 2, 3, or 4) substituents selected from the group consisting of (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo, carboxy, aryl, aryloxy, heteroaryl, and heteroaryloxy.

Another more specific value for L is a divalent, branched or unbranched, saturated or unsaturated, hydrocarbon chain, having from 3 to 15 carbon atoms, wherein one or more (e.g. 1, 2, 3, or 4) of the carbon atoms is optionally replaced by (—O—) or (—NR—).

Another more specific value for L is a divalent, branched or unbranched, saturated or unsaturated, hydrocarbon chain, having from 3 to 15 carbon atoms.

Another more specific value for L is a divalent, branched or unbranched, hydrocarbon chain, having from 3 to 15 carbon atoms.

Another more specific value for L is a divalent, branched or unbranched, hydrocarbon chain, having from 6, 7, or 8 carbon atoms.

Another more specific value for L is a divalent hydrocarbon chain having 8, 9, or 10 carbon atoms.

Another more specific value for L is a divalent hydrocarbon chain having 8 carbon atoms.

NSAIDS

Any NSAID possessing the requisite functionality to be incorporated into the backbone of a polymer as described herein is suitable for incorporation into the microspheres of the invention. Specific NSAIDS include 3-amino-4-hydroxybutyric acid, aceclofenac, alminoprofen, amfenac, bromfenac, bromosaligenin, bumadizon, carprofen, diclofenac, diflunisal, ditazol, enfenamic acid, etodolac, etofenamate, fendosal, fepradinol, flufenamic acid, gentisic acid, glucamethacin, glycol salicylate, meclofenamic acid, mefenamic acid, mesalamine, niflumic acid, olsalazine, oxaceprol, S-adenosylmethionine, salicylic acid, salsalate, sulfasalazine or tolfenamic acid. Preferred NSAIDS include salicylic acid and diflunisal. The NSAID can also be a cyclooxygenase-2 (COX-2) inhibitor, such as, e.g., celecoxib, etoricoxib, lumiracoxib, meloxicam, onconoxib, parecoxib, rofecoxib, tilmacoxib, valdecoxib, any other COX-2 inhibitor, or any combinations thereof.

Local Anesthetics

Local anesthetics suitable for mixing with the polymers include, but are not limited to, benzocaine, bupivacaine, butacaine, butanilicane, carticaine, chloroprocaine, cocaine, cyclomethycaine, dibucaine, diperocaine, etidocaine, fomocaine, isobucaine, ketamine, leucinocaine, lidocaine, lignocaine, mepivacaine, meprylcaine, myrtecaine, octacaine, oxybuprocaine, parethoxycaine, phenacaine, piperocaine, pramoxine, prilocalne, procaine, propanocaine, propoxycaine, proxymetacaine, pyrrocaine, ropivacaine, tetracaine, tolycaine, and the like (Catterall, W., Mackie, K. Local Anesthetics, Goodman & Gilman's The Pharmacological Basis of Therapeutics. 10^{th} Edition. Hardman, J. G., Limbid, L. E., Gilman, A. G., Eds. New York: McGraw-Hill, 2001).

Narcotic Analgesics

Narcotic analgesics suitable for mixing with the polymers include, but are not limited to, alfentanil, bremazocine, buprenorphine, butorphanol, codeine, CTOP, [d-Ala²] deltorphin I, [d-Ala², Glu⁴] deltorphin (deltorphin II), DADL, DALCE, DAMGO, dihydrocodeine, dihydrocodeinone, diphenoxylate, DPDPE, DSLET, dynorphin A, dynorphin B, endomorphin-1, endomorphin-2, βₕ-endorphin, FK-33824, [Leu⁵] enkephalin, [Met⁵] enkephalin, ethylketocyclazocine, etorphine, fentanyl, heroin, hydrocodone, hydromorphone, levallorphan, levorphanol, meperidine, methadone morphiceptin, morphine, morphine-6-glucuronide, nalbuphine, α-neoendorphin, β-neoendorphin, orphinan FQ/nociceptin, PL-017, oxycodone, oxymorphone, pentazocine, propoxyphene, remifentanil, spiradoline, sufentanil, tramadol, U50,488, U69,593, their metabolites, and the like (Gutstein, H. B. Akil, H. Opioid Analgesics, Goodman & Gilman's The Pharmacological Basis of Therapeutics, 10^{th} Edition, Hardman, J. G., Limbid, L. E., Gilman, A. G., Eds. New York: McGraw-Hill, 2001).

Injectable Particles

Typically, the size or shape of the particle is not critical, provided the particle is not so large that it causes discomfort or other unwanted effects following administration. Typically, particles of the invention will have a maximum dimension of less than about 1 mm and a minimum dimension of greater than about 1 nm. Preferably, particles of the invention will have a maximum dimension of less than about 100 μm and a minimum dimension of greater than 500 nm. A preferred injectable particle is a microsphere.

Microsphere Preparation

In order to mix the local anesthetic and/or narcotic analgesic drugs with the polymers, the polymers are dissolved in a suitable organic solvent, including but not limited to chloroform, methylene chloride, and other water-immiscible vehicles. The local anesthetic and/or narcotic analgesic drugs, either as free bases or salts, are dissolved in organic solvents suitable for mixing with the polymer solutions. Alternatively, the local anesthetic and/or narcotic analgesic drugs, either as free bases or salts, are dissolved directly in the polymer solutions. Alternatively, the polymers are dissolved in a suitable organic solvent, while the local anesthetic and/or narcotic analgesic drugs are dissolved in a different immiscible vehicle, such as water.

The above solutions of polymers, alone or containing local anesthetic and/or narcotic analgesic drugs, are used to prepare microspheres typically having diameters from 0.001 μm to about 100 μm by any of a number of published methods (e.g., see O'Donnell, P. B., McGinty, J. W. Preparation of microspheres by the solvent evaporation technique. Advanced Drug Delivery Reviews 1997 28:2542; O'Hara, P., Hickey, A. J. Respirable PLGA micro spheres containing rifampicin for the treatment of tuberculosis: manufacture and characterization. Pharm. Res. 2000 17:955-961; and Liggins, R. T., Burt, H. M. Paclitaxel loaded poly(L-lactic acid) microspheres: properties of microspheres made with low molecular weight polymers. Int. J. Pharmaceutics 2001 222:19-33).

Injectable Preparations

In order to prepare formulations suitable for injection, a measured amount (e.g., 100 mg) of injectable particles are added to a measured volume (e.g., 1 ml) of a suitable pharmaceutical vehicle, the contents of which may include, but are not limited to, water for injection, sodium chloride injection, Ringer's injection, lactated Ringer's injection, dextrose injection, dextrose and sodium chloride injection, benzyl alcohol, ethyl alcohol, polyethylene glycol, propylene glycol, corn oil, cottonseed oil, peanut oil, sesame oil, ethyl oleate, isopropyl myristate, benzyl benzoate, phenylmercuric nitrate, thiomersal, benzethonium chloride, benzalkonium chloride, phenol, cresol, chlorobutanol benzoic acid, p-hydroxybenzoic acid, methyl-p-hydroxybenzoate, propyl-p-hydroxybenzoate, hexylresorcinol, phenylmercuric benzoate, Polysorbate 80, SDS, SLS, TWEEN-80, mannitol, sorbitol, hydroxyethylcellulose, hydroxymethylcellulose, hydroxypropylcellulose, and the like. The injectable particles and liquid vehicle are mixed by using any number of standard methods to form a suitable formulation for injection. Prior to injection, the formulation is sterilized by using any number of standard methods.

Administration

For example, a formulation can be administered by injection to human subjects, by drawing the sterile formulation into a sterile syringe fitted with a needle of appropriate size. The area of skin through which the needle will pass is swabbed with alcohol, and a measured volume (e.g., 1 ml) of the formulation is injected into the intra-articular region of an inflamed joint or as appropriate based on the condition being treated.

The amount of the composition required for use in treatment will vary with the nature of the condition being treated and the age and condition of the patient and will be ultimately at the discretion of the attendant physician or clinician.

The ability of a composition of the invention to treat rheumatoid arthritis or other conditions may be determined using pharmacological models that are well known to the art, or may be determined using the models described in the Examples herein below.

The invention will now be illustrated by the following non-limiting Examples.

### EXAMPLES

### Example 1

### Preparation, Formulation, and In Vivo Effects of NSAID Polymer Microspheres

A biocompatible, biodegradable polymer incorporating an NSAID in the polymer backbone (e.g. salicylic acid, a salicylate derivative, or diflunisal) is prepared (for example as described by Erdmann, L., et al., Biomaterials 2000 21:1941-1946; or for example, as described in International Patent Application Publication Numbers WO 02/09767A2, WO 09768A2, and WO 99/12990).

The polymer is dissolved in anhydrous methylene chloride, and the solution is used to prepare microspheres having a mean diameter of 50 μm. The microspheres can be prepared using any suitable technique, for example, they can be prepared as described by O'Donnell, P. B., et al., Advanced Drug Delivery Reviews, 1997, 28:2542.

An aliquot (e.g., 100 mg) of dry microspheres is transferred to a disposable 3-ml syringe fitted with a disposable 3-way stopcock, and the assembly is sterilized with gamma radiation. A volume (e.g., 1 ml) of saline for injection also containing mannitol, hydroxymethylcellulose, and TWEEN-80 is drawn into another disposable 3-ml syringe, to which a disposable filter (0.22 μm pore size) is affixed. The liquid-filled syringe is attached to the sterilized syringe/stopcock assembly, and the liquid is transferred through the filter to the microsphere-filled syringe. The disposable filter is removed, the syringes are reconnected via the stopcock, and the contents are vigorously transferred back and forth to create a formulation for injection.

A standard experimental model of arthritis is created by injecting an aqueous solution of ovalbumin into the knees of rabbits (for example, see Horisawa, E., et al., Pharm. Res., 2002 19:403-410. Once chronic synovitis is established, groups of four rabbits receive single 0.1 ml i.a. injections of 1) the salicylate polymer microsphere formulation, 2) injection vehicle without polymer microspheres; or 3) injection vehicle containing betamethasone sodium phosphate (2% free base). At 3-day intervals over a 3-week period, joint swelling (circumference) is assessed, synovial fluid samples (25 μl) are collected for analysis of salicylate and betamethasone concentrations, and venous blood samples (0.5 ml) are collected for analysis of salicylate and betamethasone serum concentrations and ovalbumin antibody titers. At the end of 3 weeks the animals are sacrificed for histological assessment of joint damage.

Following the above procedure, it is anticipated that the animals receiving salicylate polymer microsphere injections will typically have high, sustained salicylate synovial fluid levels, and low salicylate serum levels, over the 3-week post-injection period. The animals receiving betamethasone injections will typically have low betamethasone levels in both synovial fluid and serum at early time points, after which betamethasone levels will be undetectable. Both the salicylate polymer microsphere- and betamethasone-treated animals will typically have significantly reduced joint swelling and serum ovalbumin antibody titers compared to vehicle controls during the 3-week period. The salicylate polymer microsphere-treated animals will typically have significantly less damage to the injected knee joints than the animals receiving betamethasone.

### Example 2

### Preparation, Formulation, and In Vivo Effects of NSAID Polymer Microspheres Containing Local Anesthetic

A biocompatible, biodegradable polymer incorporating an NSAID in the polymer backbone can be prepared as described in Example 1.

The polymer is dissolved in anhydrous methylene chloride, and the solution is used together with an aqueous solution of lidocaine hydrochloride to prepare microspheres containing 1% lidocaine (free base) by weight, and having a mean diameter of 50 μm; the microspheres can be prepared, for example, as described in Example 1.

An aliquot (e.g., 100 mg) of dry microspheres is transferred to a disposable 3-ml syringe fitted with a disposable 3-way stopcock, and the assembly is sterilized with gamma radiation. A volume (e.g., 1 ml) of saline for injection also containing mannitol, hydroxymethylcellulose, and TWEEN-80 is drawn into another disposable 3-ml syringe, to which a disposable filter (0.22 μM pore size) is affixed. The liquid-filled syringe is attached to the sterilized syringe/stopcock assembly, and the liquid is transferred through the filter to the microsphere-filled syringe. The disposable filter is removed, the syringes are reconnected via the stopcock, and the contents are vigorously transferred back and forth to create a formulation for injection.

A standard experimental model of arthritis is created by injecting an aqueous solution of ovalbumin into the knees of rabbits (for example, see Horisawa, E., et al., Pharm. Res., 2002 19:403-410. Once chronic synovitis is established, groups of four rabbits receive single 0.1 ml i.a. injections of 1) the salicylate polymer/lidocaine microsphere formulation; 2) injection vehicle without salicylate polymer/lidocaine microspheres; or 3) injection vehicle containing lidocaine hydrochloride (1% free base). At 3-day intervals over a 3-week period, joint swelling (circumference) was assessed, synovial fluid samples (25 μl) were collected for analysis of salicylate and lidocaine concentrations, and venous blood samples (0.5 ml) were collected for analysis of salicylate and lidocaine serum concentrations and ovalbumin antibody titers. At the end of 3 weeks the animals were sacrificed for histological assessment of joint damage.

Following the above procedure, it is anticipated that the animals receiving salicylate polymer/lidocaine microsphere injections will typically have high, sustained salicylate synovial fluid levels, and low salicylate serum levels, over the 3-week post-injection period. Lidocaine will typically not be detectable in synovial fluid or serum of any animal at any time point. The salicylate polymer/lidocaine microsphere-treated animals will typically have significantly reduced joint swelling and serum ovalbumin antibody titers compared to both lidocaine and vehicle controls during the 3-week period.

### Example 3

### Preparation, Formulation, and In Vivo Effects of NSAID Polymer Microspheres Containing Narcotic Analgesic

A biocompatible, biodegradable polymer incorporating an NSAID in the polymer backbone can be prepared as described in Example 1.

The polymer is dissolved in anhydrous methylene chloride, and the solution is used together with an aqueous solution of morphine sulfate to prepare microspheres containing 2% morphine (free base) by weight, and having a mean diameter of 50 μm; the microspheres can be prepared, for example, as described in Example 1.

An aliquot (e.g., 100 mg) of the dry microspheres is transferred to a disposable 3-ml syringe fitted with a disposable 3-way stopcock, and the assembly is sterilized with gamma radiation. A volume (e.g., 1 ml) of saline for injection also containing mannitol, hydroxymethylcellulose, and TWEEN-80 is drawn into another disposable 3-ml syringe, to which a disposable filter (0.22 μM pore size) was affixed. The liquid-filled syringe is attached to the sterilized syringe/stopcock assembly, and the liquid is transferred through the filter to the microsphere-filled syringe. The disposable filter is removed, the syringes are reconnected via the stopcock, and the contents are vigorously transferred back and forth to create a formulation for injection.

A standard experimental model of arthritis is created by injecting an aqueous solution of ovalbumin into the knees of rabbits (for example, see Horisawa, E., et al., Pharm. Res., 2002 19:403-410. Once chronic synovitis is established, groups of four rabbits receive single 0.1 ml i.a. injections of 1) the salicylate polymer/morphine microsphere formulation; 2) injection vehicle without salicylate polymer/morphine microspheres; or 3) injection vehicle containing morphine sulfate (2% free base). At 3-day intervals over a 3-week period, joint swelling (circumference) is assessed, synovial fluid samples (25 μl) are collected for analysis of salicylate and morphine concentrations, and venous blood samples (0.5 ml) are collected for analysis of salicylate and morphine serum concentrations and ovalbumin antibody titers. At the end of 3 weeks the animals are sacrificed for histological assessment of joint damage.

Following the above procedure, it is anticipated that the animals receiving salicylate polymer/morphine microsphere injections will typically have high, sustained salicylate and morphine synovial fluid levels, and low salicylate and morphine serum levels, over the 3-week post-injection period. The animals receiving morphine injections will typically have undetectable levels of morphine in synovial fluid and serum at every time point. The salicylate polymer/morphine microsphere-treated animals will typically have significantly reduced joint swelling and serum ovalbumin antibody titers compared to vehicle and morphine controls during the 3-week period.

### Example 4

### Preparation, Formulation, and In Vivo Effects of NSAID Polymer Microspheres Containing Local Anesthetic and Narcotic Analgesic

A biocompatible, biodegradable polymer incorporating an NSAID in the polymer backbone can be prepared as described in Example 1.

The polymer is dissolved in anhydrous methylene chloride, and the solution is used together with an aqueous solution of lidocaine hydrochloride and morphine sulfate to prepare microspheres containing 1% lidocaine and 2% morphine (free bases) by weight, and having a mean diameter of 50 μm; the microspheres can be prepared, for example, as described in Example 1.

An aliquot (e.g., 100 mg) of the dry microspheres is transferred to a disposable 3-ml syringe fitted with a disposable 3-way stopcock, and the assembly is sterilized by using, e.g., gamma radiation. A volume (e.g., 1 ml) of saline for injection also containing mannitol, hydroxymethylcellulose, and TWEEN-80 is drawn into another disposable 3-ml syringe, to which a disposable filter (0.22 μm pore size) is affixed. The liquid-filled syringe is attached to the sterilized syringe/stopcock assembly, and the liquid is transferred through the filter to the microsphere-filled syringe. The disposable filter is removed, the syringes were reconnected via the stopcock, and the contents are vigorously transferred back and forth to create a formulation for injection.

A standard experimental model of arthritis is created by injecting an aqueous solution of ovalbumin into the knees of rabbits (for example, see Horisawa, E., et al., Pharm. Res., 2002 19:403-410. Once chronic synovitis was established, groups of four rabbits received single 0.1 ml i.a. injections of 1) the salicylate polymer/lidocaine/morphine microsphere formulation; 2) injection vehicle without salicylate polymer/lidocaine/morphine microspheres; 3) injection vehicle containing lidocaine hydrochloride (1% free base); or 4) injection vehicle containing morphine sulfate (2% free base). At 3-day intervals over a 3-week period, joint swelling (circumference) is assessed, synovial fluid samples (25 μl) were collected for analysis of salicylate, lidocaine, and morphine concentrations, and venous blood samples (0.5 ml) are collected for analysis of salicylate, lidocaine, and morphine serum concentrations and ovalbumin antibody titers. At the end of 3 weeks the animals are sacrificed for histological assessment of joint damage.

Following the above procedure, it is anticipated that the animals receiving salicylate polymer/lidocaine/morphine microsphere injections will typically have high, sustained synovial fluid levels of salicylate and morphine, and very low blood levels of salicylate and morphine, over the 3-week treatment period. Lidocaine will typically be undetectable in synovial fluid and serum of these animals at any time point. The animals receiving injections of lidocaine or morphine will typically have undetectable levels of either drug in synovial fluid and serum at any time point. The salicylate polymer/lidocaine/morphine microsphere-treated animals will typically show significantly reduced joint swelling and serum ovalbumin antibody titers compared to vehicle, lidocaine, and morphine controls during the 3-week treatment period.

### Example 5

### Representative Injectable Dosage Forms, Comprising Injectable Particles of the Invention (‘Injectable Particles ’), for Therapeutic Use in Humans

| (i) Injection 1 (1 mg/ml) | mg/ml | |
|---|---|---|
| ‘injectable particles’ | 1.0 | |
| Dibasic sodium phosphate | 12.0 | |
| Monobasic sodium phosphate | 0.7 | |
| Sodium chloride | 4.5 | |
| 1.0 N Sodium hydroxide solution | q.s. | |
| (pH adjustment to 7.0-7.5) | | |
| Water for injection | q.s. ad 1 | mL |

| (ii) Injection 2 (10 mg/ml) | mg/ml | |
|---|---|---|
| ‘injectable particles’ | 10.0 | |
| Monobasic sodium phosphate | 0.3 | |
| Dibasic sodium phosphate | 1.1 | |
| Polyethylene glycol 400 | 200.0 | |
| 01 N Sodium hydroxide solution | q.s. | |
| (pH adjustment to 7.0-7.5) | | |
| Water for injection | q.s. ad 1 | mL |

The above formulations may be obtained by conventional procedures well known in the pharmaceutical art.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1-66. (canceled)

67. An injectable particle(s), that comprise(s) a biodegradable polymer(s); and
one or more of an NSAID(s), a local anesthetic(s) or a narcotic analgesic(s).

68. The particle(s) of claim 1, comprising a biodegradable polymer(s), and an NSAID(s).

69. The particle(s) of claim 2, further comprising a local anesthetic(s).

70. The particle(s) of claim 2, further comprising a narcotic analgesic(s).

71. The particle(s) of claim 4, further comprising a local anesthetic(s).

72. The particle(s) of claim 1, wherein the NSAID(s) is released, upon hydrolysis, from the polymer(s).

73. The particle(s) of claim 1, wherein local anesthetic and a narcotic analgesic are released, upon hydrolysis, from the polymer(s).

74. The particle(s) of claim 1, wherein the polymer(s) comprise(s) an anhydride, ester, thioester, amide, anhydride, carbonate or carbamate polymer(s).

75. The particle(s) of claim 1, wherein the polymer(s) comprise(s) one or more units of chemical formula
R₁-A-L-A- (1),
R₂-A-L-A-R₃-A-L-A- (II), or
—C(═O)R₄—X-L-X—R₄—C(═O)—O— (II)
wherein
each R₁, R₂, R₃ or R₄, independently, comprises one or more NSAID(s);
each A or X, independently, comprises ester, thioester, or amide; and
each L, independently, comprises a linker.

76. The particle(s) of claim 8, wherein each L, independently, comprises an aliphatic or aromatic linker.

77. The particle(s) of claim 8, wherein each L, independently, comprises a biologically active linker.

78. The particle(s) of claim 1, wherein each aliphatic or aromatic linker, independently, comprises a divalent (C₁-C₂₅) hydrocarbon, which may be branched, unsaturated, or substituted by (—O—), (—NR—), (C₁-C₆) alkoxy, (C₃-C₆) cycloalkyl, (C₁-C₆) alkanoyl, (C₁-C₆) alkanoyloxy, (C₁-C₆) alkoxycarbonyl, (C₁-C₆) alkylthio, azido, cyano, nitro, halo, hydroxy, oxo, carboxy, aryl, aryloxy, heteroaryl or heteroaryloxy.

79. The particle(s) of claim 8, wherein each L, independently, comprises a divalent C₆, C₇, C₈, C₉ or C₁₀ hydrocarbon.

80. The particle(s) of claim 8, wherein each L, independently, comprises about 25 dalton to about 400 dalton.

81. The particle(s) of claim 8, wherein each L, independently, comprises a divalent (C₁-C₂₅) hydrocarbon, which may be branched, unsaturated or substituted by (—O—), (—NR—), (C₁-C₆)alkoxy, (C₃-C₆) cycloalkyl, (C₁-C₆) alkanoyl, (C₁-C₆) alkanoyloxy, (C₁-C₆) alkoxycarbonyl, (C₁-C₆) alkylthio, mercapto, amine, carboxyl, azido, cyano, nitro, halo, hydroxy, oxo, carboxy, aryl, aryloxy, heteroaryl or heteroaryloxy.

82. The particle(s) of claim 8, wherein each L, independently, comprises a peptide(s) or an amino acid(s).

83. The particle(s) of claim 8, wherein each L, independently, comprises a divalent (C₃-C₁₅) hydrocarbon, which may be branched, unsaturated or substituted by (—O—), (—NR—), (C₁-C₆) alkoxy, (C₃-C₆) cycloalkyl, (C₁-C₆) alkanoyl, (C₁-C₆) alkanoyloxy, (C₁-C₆) alkoxycarbonyl, (C₁-C₆) alkylthio, mercapto, amine, carboxyl, azido, cyano, nitro, halo, hydroxy, oxo, carboxy, aryl, aryloxy, heteroaryl or heteroaryloxy.

84. The particle(s) of claim 16, wherein each L substituent(s), independently, is(are) selected for modifying the polymer(s) properties by branching, cross-linking or appending one or more biologically or pharmaceutically active molecule(s).

85. The particle(s) of claim 16, wherein each L substituent(s), independently, is(are) selected for changing the polymer solubility, or affecting the polymer(s) biodistribution upon administration or application.

86. The particle(s) of claim 1, wherein the NSAID(s), independently, comprise(s) 3-amino-4-hydroxybutyric acid, aceclofenac, alminoprofen, amfenac, bromfenac, bromosaligenin, bumadizon, carprofen, diclofenac, diflunisal, ditazol, enfenamic acid, etodolac, etofenamate, fendosal, fepradinol, flufenamic acid, gentisic acid, glucamethacin, glycol salicylate, meclofenamic acid, mefenamic acid, mesalamine, niflumic acid, olsalazine, oxaceprol, S-adenosylmethionine, salicylic acid, salsalate, sulfasalazine or tolfenamic acid.

87. The particle(s) of claim 1, wherein the NSAID(s) comprise(s) a cyclooxygenase-2 (COX-2) enzyme inhibitor(s).

88. The particle(s) of claim 1, wherein the COX-2 enzyme inhibitor(s). comprise(s) one or more of celecoxib, etoricoxib, lumiracoxib, meloxicam, onconoxib, parecoxib, rofecoxib, tilmacoxib or valdecoxib.

89. The particle(s) of claim 1, wherein the NSAID(s) comprise(s) salicylic acid.

90. The particle(s) of claim 1, wherein the local anesthetic(s), independently, comprise(s) benzocaine, bupivacaine, butacaine, butanilicane, carticaine, chloroprocaine, cocaine, cyclomethycaine, dibucaine, diperocaine, etidocaine, fomocaine, isobucaine, ketamine, leucinocaine, lidocaine, lignocaine, mepivacaine, meprylcaine, myrtecaine, octacaine, oxybuprocaine, parethoxycaine, phenacaine, piperocaine, pramoxine, prilocalne, procaine, propanocaine, propoxycaine, proxymetacaine, pyrrocaine, ropivacaine, tetracaine or tolycaine.

91. The particle(s) of claim 1, wherein the narcotic analgesic(s), independently, comprise(s) alfentanil, bremazocine, buprenorphine, butorphanol, codeine, CTOP, [d-Ala²] deltorphin I, [d-Ala², Glu⁴] deltorphin (deltorphin II), DADL, DALCE, DAMGO, dihydrocodeine, dihydrocodeinone, diphenoxylate, DPDPE, DSLET, dynorphin A, dynorphin B, endomorphin-1, endomorphin-2, βₕ-endorphin, FK-33824, [Leu⁵] enkephalin, [Met⁵] enkephalin, ethylketocyclazocine, etorphine, fentanyl, heroin, hydrocodone, hydromorphone, levallorphan, levorphanol, meperidine, methadone, morphiceptin, morphine, morphine-6-glucuronide, nalbuphine, α-neoendorphin, β-neoendorphin, orphinan FQ/nociceptin, PL-017, oxycodone, oxymorphone, pentazocine, propoxyphene, remifentanil, spiradoline, sufentanil, tramadol, U50,488 or U69,593.

92. The particle(s) of claim 1, wherein the local anesthetic(s) comprise(s) lidocaine.

93. The particle(s) of claim 1, wherein the local anesthetic(s), independently, is(are) present in an amount of about 0.1% wt/wt to about 10% wt/wt.

94. The particle(s) of claim 1, wherein the local anesthetic(s), independently, comprise(s) a free base(s) or a pharmaceutical salt(s) thereof.

95. The particle(s) of claim 27, wherein the pharmaceutical salt(s) of the local anesthetic(s), independently, comprise(s) sulfate, phosphate, acetate, tartrate or hydrochloride.

96. The particle(s) of claims 1, wherein the narcotic analgesic(s) comprise(s) morphine.

97. The particle(s) of claim 1, wherein the narcotic analgesic(s), independently, is(are) present in in an amount of about 1% wt/wt to about 30% wt/wt.

98. The particle(s) of claim 1, wherein the narcotic analgesic(s), independently, is(are) present as a free base, or as a pharmaceutical salt(s).

99. The particle(s) of claim 31, wherein the pharmaceutical salt(s) of the narcotic analgesic(s), independently, comprise(s) sulfate, phosphate, acetate, tartrate or hydrochloride.

100. The particle(s) of claim 1, comprising about 0.001 micron to about 100 micron particle(s) as determined by dynamic light scattering.

101. The particle(s) of claim 1, which comprise a microsphere(s).

102. The microsphere(s) of claim 34, comprising about 0.001 micron to about 100 micron particle(s) as determined by dynamic light scattering.

103. A microsphere(s), comprising a polymer(s) that upon hydrolysis releases one or more of salicylic acid or diflunisal.

104. The microsphere(s) of claim 36, further comprising lidocaine or morphine.

105. The microsphere(s) of claim 36, further comprising lidocaine.

106. The microsphere(s) of claim 36, further comprising morphine.

107. The microsphere(s) of claim 39, further comprising lidocaine.

108. A pharmaceutical composition, comprising a plurality of the particles of claim 1, and a pharmaceutically acceptable carrier.

109. The composition of claim 41, comprising a systemic formulation.

110. The composition of claim 41, comprising an injectable formulation.

111. The composition of claim 41, comprising a site-of-pain or site-of-inflammation formulation.

112. A method of treating pain or inflammation or to prevent or reduce swelling, or a condition associated with pain, inflammation or swelling at a site, comprising administering or applying to the site an analgesic, anti-inflammatory or anti-swelling effective amount of the particle(s) of claim 1 or composition or formulation thereof.

113. The method of claim 45, wherein the pain or inflammation or condition associated with pain or inflammation comprises spinal stenosis, bursitis, tendonitis, epicondylitis, fibromyalgia, chronic foot and ankle pain, calcaneal spur syndrome, neuralgia, metatarsalgia, metatarsophalangeal articulation, rheumatoid arthritis or osteoarthritis.

114. The method of claim 45, wherein the swelling is associated with central nervous system tissue.

115. The method of claim 45, wherein the administration or application is conducted systemically, or by injection.

116. The method of claim 45, wherein the injection is conducted locally at or near the site.

117. The method of claim 45, wherein the inflammation is associated with the nervous system or surrounding tissue.

118. The method of claim 50, wherein the nervous system tissue inflammation is associated with or following injury.
